(19) United States
(12) Patent Application Publication
(10) Pub. No.: US 2018/0279943 A1
(43) Pub. Date: Oct. 4, 2018

(54) SYSTEM AND METHOD FOR THE ANALYSIS AND TRANSMISSION OF DATA, IMAGES AND VIDEO RELATING TO MAMMALIAN SKIN DAMAGE CONDITIONS

(71) Applicant: Tissue Analytics, Inc., Baltimore, MD (US)

(72) Inventors: Joshua BUDMAN, Baltimore, MD (US); Kevin P. KEENAHAN, Baltimore, MD (US); Gabriel A. BRAT, Brookline, MA (US)

(73) Assignee: Tissue Analytics, Inc., Baltimore, MD (US)

(21) Appl. No.: 15/521,954

(22) PCT Filed: Oct. 26, 2015
(86) PCT No.: PCT/US2015/057344

§ 371 Date: Apr. 26, 2017

Related U.S. Application Data
(60) Provisional application No. 62/069,993, filed on Oct. 29, 2014.
(60) Provisional application No. 62/069,972, filed on Oct. 29, 2014.

(51) Int. Cl. A61B 5/00; G06T 7/00; G06T 7/11; G06T 7/90

(52) U.S. Cl. CPC A61B 5/445; G06T 7/0014; G06T 2207/30088; G06T 7/97; G06T 7/90; G06T 7/11

(57) ABSTRACT

Data, images and video characterizing mammalian skin damage conditions are collected and analyzed in part using a mobile device as a data collection engine at the point of care. The device establishes communications with a server where the information is stored in a database. The server has an image analysis component applying image processing and analysis techniques, the results of which are reported to the initial data collection engine and made available at a central web portal where users can view the data as well as trends in the data. The central web portal is equipped with a billing unit and portal by which users can generate reimbursement requests. The system has a predictive analysis component that produces predictions based on the data in the database, and predicts the probable progress of the skin damage condition. The predictive analysis is also available to users of the central web portal.

### CROSS REFERENCE TO RELATED APPLICATIONS

This application claims the priority of Provisional U.S. Patent Application Ser. No. 62/069,972, filed Oct. 29, 2014; and Ser. No. 62/069,993, filed Oct. 29, 2014, which applications are hereby incorporated by reference, in their entireties.

### FIELD OF THE INVENTION

The present invention is directed at developing a system that captures data, an image or images and a video of a human skin damage condition at the point of care, analyzes the image(s) and video in an automated fashion and transmits the data, image(s) and video with the analysis to a central location.

### DESCRIPTION OF THE RELATED ART

In order to measure the status of a skin condition, practitioners current rely on the use of rulers or naked eye approximations. Studies have shown that for a particular condition, chronic wounds, these techniques have 45% error. (See, Measuring wound length, width, and area: which technique? Langemo, Anderson, Hanson, Hunter, Thompson, Advances in Skin & Wound Care, January 2008, 21(1): 42-45 1879-1882.)

In addition, literature reports that these techniques have an inter-rater error, i.e. the error that occurs between two separate individuals measuring the same condition, of 16-50%. (See, Reproducibility of Current Wound Surface Measurement, Koel, Gerard, and Frits Oosterveld, European Wound Management Conference Proceeding (2008). This number is elevated by the fact that patients with skin conditions often have care provided for them in a variety of settings by a variety of providers. All of this makes it very difficult for providers to accurately track the longitudinal progress of these conditions.

Some existing devices or systems have been developed in order to address this problem. The Mobile Wound Management Tool by WoundMatrix combines a point-of-care smartphone application with a server-hosted web environment to address providers' inability to appropriately document wounds and track changes over time. WoundMatrix's system, however, does not provide advanced and automated analytics to standardize measurements and instead relies on the provider's judgment to perform these measurements manually. Additionally, this method still requires the presence of a ruler to conduct these measurements. Finally, while WoundMatrix does obtain information about a wound's location on a patient's body, it does not gather information regarding other aspects of the patient's treatment and thus is unable to assist providers in detecting the efficacy of current treatments.

Healogram provides a system that collects patient photographs and data at the point of care and relays this information to clinicians at a centralized portal. Healogram also provides longitudinal tracking capabilities by overlaying an old image of a wound over the camera screen before taking the new image. Similar to WoundMatrix, however, Healogram does not have automated image analysis capabilities and does not directly improve the accuracy of wound measurement and characterization. Healogram instead focuses on effective care coordination and patient compliance.

Recently, there has been development in image-based measurement from the New Zealand-based company Aranz with their Silhouette System. Silhouette's system includes smart software for measuring skin conditions such as wounds using data in both the infrared (IR) and visible ranges. The overall cost of the Silhouette System is close to $6,000 US Dollars in part due to its reliance on IR data and has thus not been widely adopted in a clinical setting.

Another image-based measurement system is the WoundMAP PUMP by MobileHealthWare. This device relies on the placement of a ruler next to the wound and allows individuals to manually locate the edges of a skin condition and compare them to the dimensions on the ruler. This system is subject to the same deficiencies as measuring skin conditions with a ruler as it approximates the skin condition as a square.

Another system that attempts to improve documentation is WoundRounds by Telemedicine, LLC. WoundRounds is a standalone device with the capability to integrate with the electronic medical record (EMR) to facilitate in-facility wound documentation. Like the prior solutions described, this system does not have advanced and automatic image analysis capabilities. Additionally, the solution relies on a cumbersome device and thus is not suitable for use on patients in settings peripheral to the wound clinic.

There are other smartphone applications that collect photographs of skin conditions but do not include photo transmission to a centralized location nor do they include image analysis capabilities. Examples of such applications include First Derm, which provides anonymous dermatology advice upon collection of a photograph, and Doctor Mole, which is an app that assesses moles and determines whether or not they are cancerous based on photographs taken at the point of care. Neither of these applications provides a photograph transmission platform nor do they have video analysis capabilities.

A final image-based measurement system is the Mobile Wound Analyzer (MOWA) by HealthPath. This is a mobile system that segments tissues within a skin condition. This system does not have edge detection capabilities, however, and relies on a user to manually detect and illustrate the edges of the skin condition.

Furthermore, no commercial methods exist to perform a blood flow analysis and full 3D reconstruction of a skin condition without any external attachments to the device collecting the digital images. Finally, no other existing commercial applications possess a fully device agnostic way to consistently longitudinally track images of a skin condition.

### SUMMARY

This disclosure is not limited to the particular systems, devices and methods described as these may vary. The terminology used in the description is for the purpose describing the particular version or embodiments only, and is not intended to limit the scope.

As used in this document, the singular forms “a”, “an”, and “the” include plural references unless the context clearly dictates otherwise. Unless defined otherwise, all technical and scientific terms used herein have the same meanings as commonly understood by one of ordinary skill in the art. Nothing in this document is to be construed as an admission that the embodiments described in this document are not entitled to antedate such disclosure by virtue of prior invention. As used in this document, the term “comprising” means “including, but not limited to”.

In one general respect, the embodiments disclose a system or method of collecting an image, video of and data about a human skin damage condition at the point of care, including but not limited to chronic wounds, acute wounds, burns, lesions, scars, psoriasis, eczema, acne, melanoma, rosacea, scabies, carcinoma, vitiligo, arrhythymia, dermatitis, keratosis, bug bites, rash, keloids, lupus, herpes, cellulitis and gonorrhea.

In another general respect, the embodiments disclose a method for measuring the surface area of the specific skin condition and characterizing the exact tissues present as evoked by the onset of the skin condition using a set reference object. The system is composed of a database of images possessing the same skin condition as the image being analyzed.

In another general respect, the embodiments disclose a system or method of analyzing the aforementioned image and video. Types of analysis provided comprise the aforementioned analysis including surface area, tissue composition of the skin condition blood flow (perfusion) profile of the skin condition and the area around the skin condition and a 3D reconstruction of the skin condition leading to a total volume calculation.

In another general respect, the embodiments disclose a system or method of transporting the analyzed image and video and associated patient data to a centralized location so that it can be analyzed by a specialist.

In another general respect, the embodiments disclose a system for displaying trends in the output of the image and video analysis at a centralized portal, preferably on the World Wide Web.

In another general respect, the embodiments disclose a system or method of correlating the image and video data with data about the patient's treatment at a central portal and a method to display the output of this correlation at this central portal to inform clinical decision making.

In another general respect, the embodiments disclose a method for allowing individuals of x to inform the system's own ability to characterize skin conditions' perfusion by using existing data from a Laser Doppler Imaging device.

### BRIEF DESCRIPTION OF THE DRAWINGS

FIG. 1 illustrates the exemplary flow for the entire system including the point-of-care data collection device, image analysis node, server-hosted database and central portal.

FIG. 2 illustrates the system's customization and tuning of the image acquisition hardware to optimize image pre-processing and standardize image registration.

FIG. 3 illustrates an exemplary object being placed next to the photographed skin condition such that said object can be referenced as a ground truth in the image.

FIG. 4 illustrates an exemplary flow for the standardization of image registration by using the known parameters of the aforementioned reference object.

FIG. 5 illustrates the exemplary flow for the method to acquire the skin condition's exact edges and tissue composition and calculate precise values for these fields.

FIG. 6 illustrates the exemplary flow for the method to combine different edge detection mechanisms for identifying the precise skin condition boundary and segment the tissues within said skin condition.

FIG. 7 illustrates screenshots of an exemplary result of the 3D reconstruction of a skin condition (pictured at the top).

FIG. 8 illustrates screenshot of an exemplary result of the perfusion monitoring of a skin condition.

FIG. 9 illustrates the exemplary flow for the system to collect data, images and videos about a patient skin condition at the point of care, transmit this information to a central location and pull back the information post-processing.

FIG. 10 illustrates the exemplary design for a web portal where providers can view the longitudinal progress of a patient's skin condition.

FIG. 11 illustrates screenshots of the exemplary design for the component that allows providers to bill for using the web portal.

FIG. 12 illustrates the exemplary flow for the system component that processes data at the database and provides predictive analysis.

### DETAILED DESCRIPTION

As used in this document, the terms “skin condition” or “skin damage condition” refer to but are not limited to chronic wounds, acute wounds, burns, lesions, scars, psoriasis, eczema, acne, melanoma, rosacea, scabies, carcinoma, vitiligo, arrhythymia, dermatitis, keratosis, bug bites, rash, keloids, lupus, herpes, cellulitis and gonorrhea.

As used in this document, the terms “image” or “medical image” refer to an electromagnetic image of a skin condition as described above.

As used in this document, the terms “patient” or “subject” refer to any subject that would be classified as a mammal.

As used in this document, the term “video” describes a set of images as described above collected in rapid succession.

As used in this document, the terms “analysis” or “image analysis” describes automated detection of the edges of a skin condition, total area calculation of the skin condition, segmentation of the tissues within the skin condition and segmentation analysis of the tissues within the skin condition.

As used in this document, the term “video analysis” describes analysis of perfusion in and around the skin condition and 3D reconstruction of the skin condition including depth and volume calculation.

As used in this document, the term “data collection engine” describes an application on any mobile device that is able to gather images and videos. This list comprises applications for mobile phones and tablets.

The present invention relates to a method or system, including a mobile phone component, a server component and a web-based component, for collecting data, photographs and videos and transmitting them to a central location.

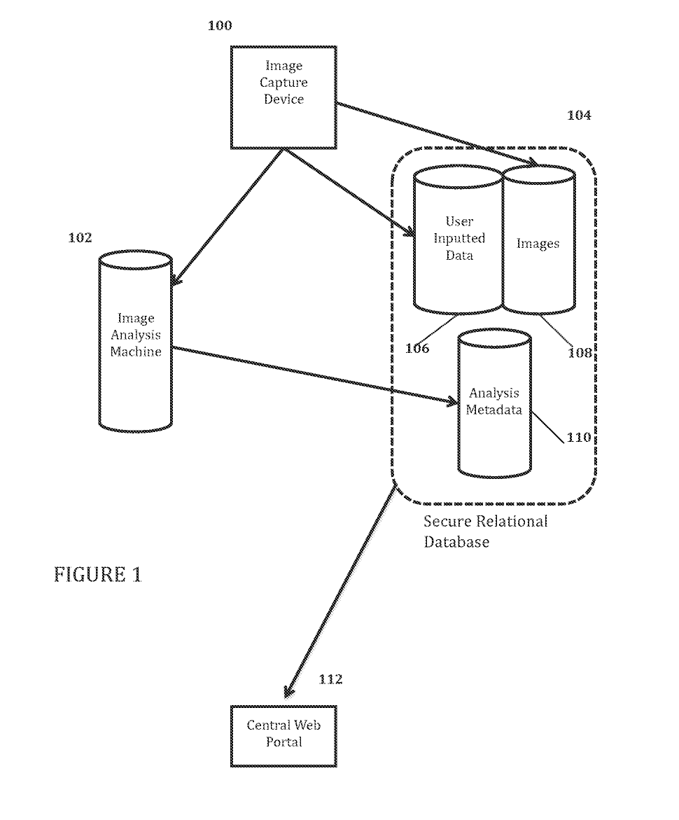

Photographs and videos are stored in a secure server storage area 104 in FIG. 1 from where they are hosted on the central portal 112 in FIG. 1.

The system provides a server node or nodes 102 in FIG. 1 to perform automated image analysis and video analysis of the images and video collected by the point-of-care data collection engine 100 in FIG. 1. This analysis is then sent with the appropriate image and video to the central web portal 108 in FIG. 1.

The system includes a database or data structure 104 in FIG. 1 that assembles patient data collected by the data collection engine 100 and matches this data with the appropriate video and images collected by 100 and stored in 104.

The image can be acquired by any device that has the ability to collect images. There are no resolution requirements on the image that is analyzed by the system described.

The system collects a set of manual, human inputs prior to analyzing the image or video. These inputs include aspects of the wound that cannot be collected using a digital image including but not limited to drainage, odor and pain.

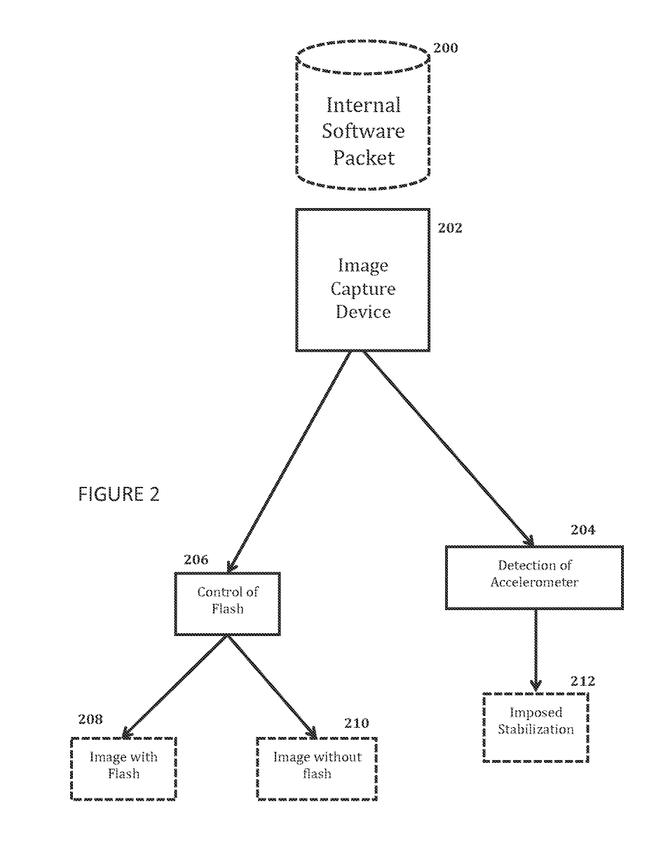

The image capture device is equipped with a software packet 200 in FIG. 2 that is able to tune the hardware to optimize image acquisition and registration.

While the image acquisition component does not require flash capabilities, if the image acquisition component has these capabilities the software packet 200 in FIG. 2 automatically acquires a pair of images—one with the flash and one without—as in 206-210 of FIG. 2.

The software packet 200 in FIG. 2 is also able to detect the device accelerometer outputs if applicable as in 204 of FIG. 2 and will acquire an image only if user motion is under a certain threshold, thus imposing stabilization as in 212 of FIG. 2.

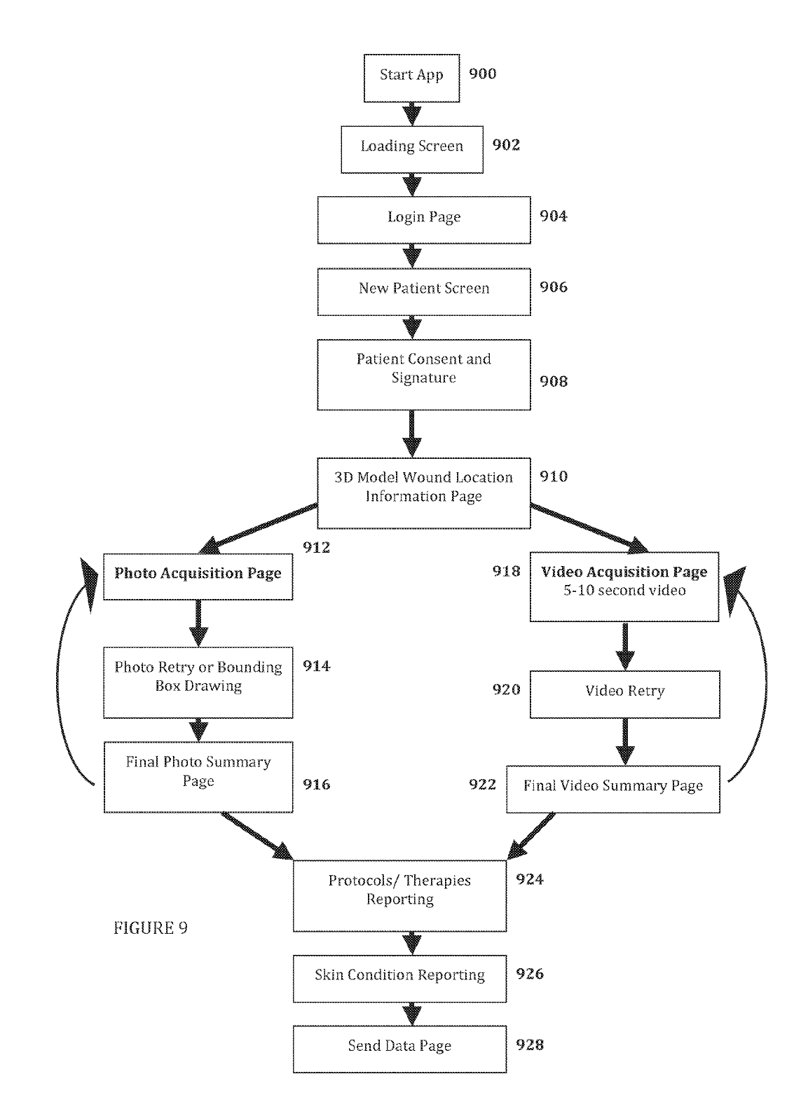

While the image analysis system does not require any user inputs, the system provides the ability to create a bounding box on the image 914 of FIG. 9 to provide ground truth foreground-background pre-processing.

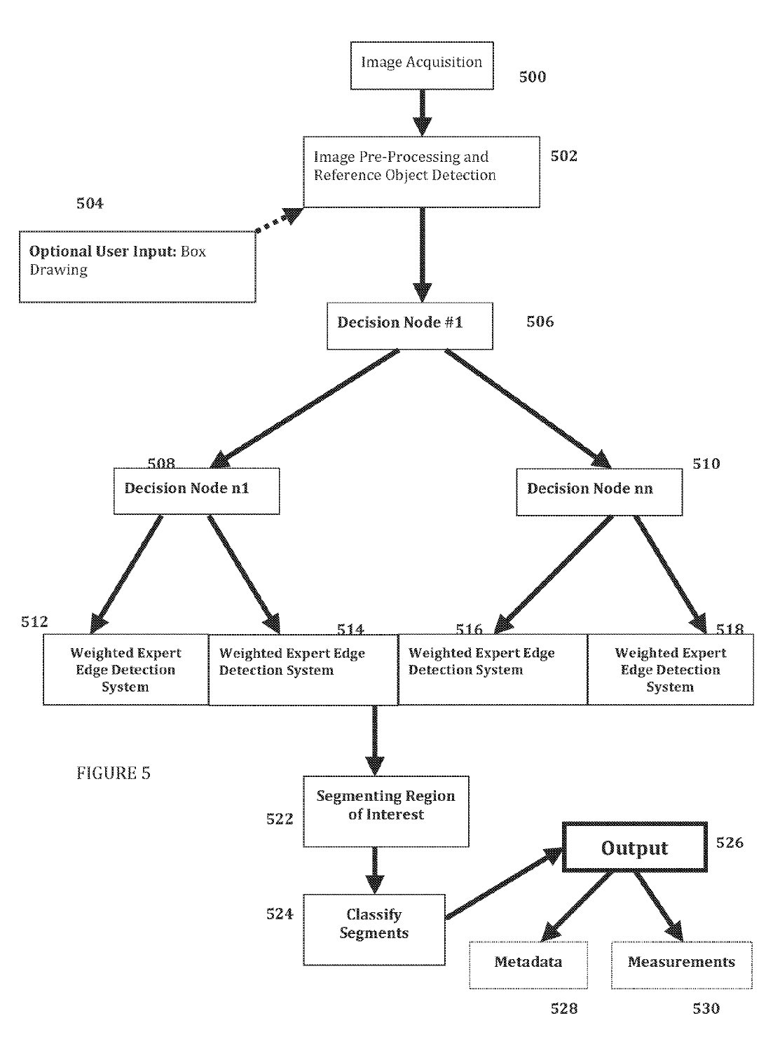

Once the image is acquired, a set of pre-processing steps take place as shown in 502 of FIG. 5. The pre-processing procedure includes erosion, smoothing and dilation of the image with a small, circular structural element to smoothen the image and remove shape artifacts.

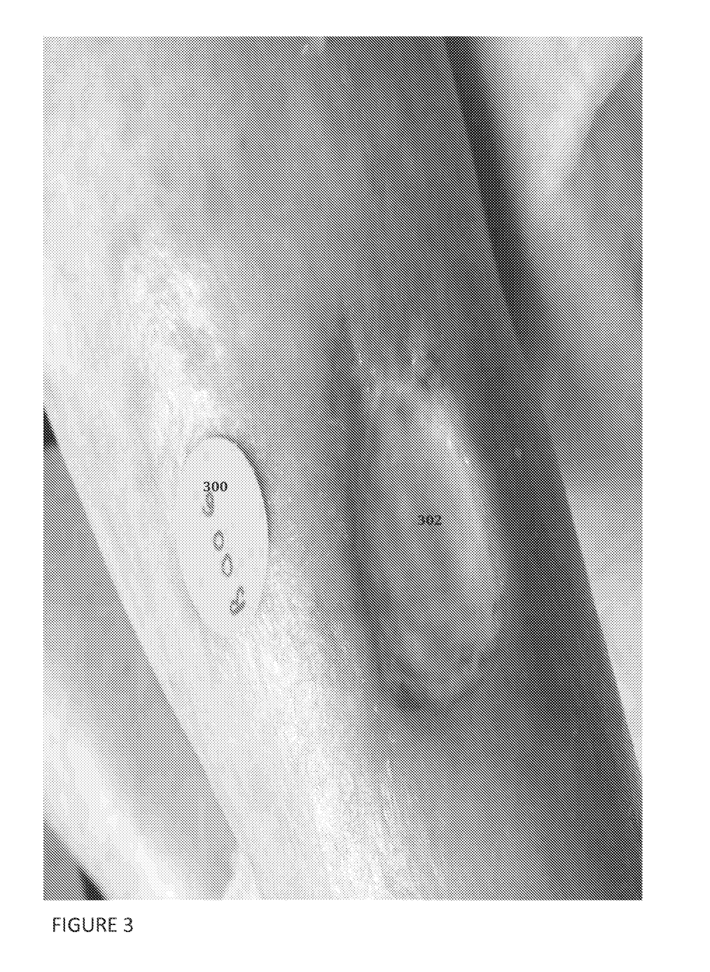

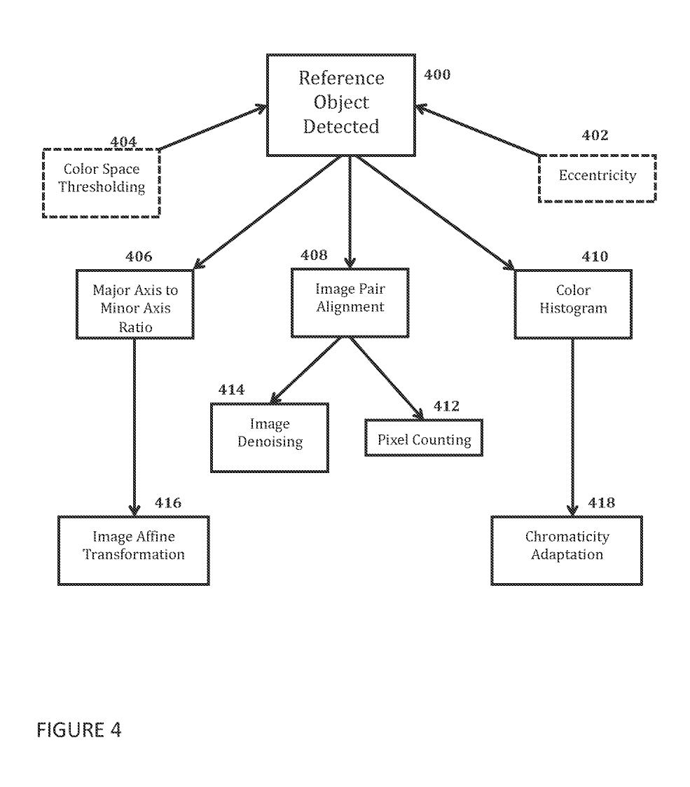

The reference object 300 in FIG. 3 allows for ground truth parameter normalization. The reference object is detected in the frame of the image in an automated fashion using a cascade of adaptive color thresholding and eccentricity detection as shown in 400-404 of FIG. 4.

As the aforementioned reference object has a known, constant cyan-magenta-yellow-key (CMYK) value color constancy algorithms can be applied to the wound images to standardize the lighting registered as in 410 and 418 of FIG. 4. These color constancy algorithms include but are not limited to the Bradford Chromaticity Adaptation Transform (Bradford CAT), Von Kries Algorithm, white balancing and the Sharp Transform.

The flash-no-flash image pair allows for automated luminance calibration by standardizing the mean value in YCbCr color space by changing the scaling parameters on the aggregation of the image pair as in 408 of FIG. 4. The image pair also allows for image denoising by performing a joint bilateral filter using the combined output of the image pair as in 414 of FIG. 4.

The reference object 300 of FIG. 3 allows for distance normalization due to the unchanging size of the aforementioned reference object. Knowing both the relative size of the skin condition and the size of reference object in the acquired image, the true size of the skin condition can be calculated by dividing the pixels within the skin condition's mask by the pixels within the reference object's mask and multiplying this ratio by the true size of the reference object such as is done in digital planimetry. The wound mask, like the reference object, is found in a fully automated fashion, which will be described in a later portion.

The reference object 300 of FIG. 3 allows for camera angle correction due to the aforementioned object's unchanging shape. Specifically, the unchanging, ground truth ratio between the major and minor axis of said reference object allows the software to perform an affine transformation on the full image prior to registration as in 416 of FIG. 4. This transformation standardizes the angle of the registered image, regardless of the user-defined angle of the camera upon initial collection of the image, thus avoiding any angled-based errors in true value calculation.

The reference object 300 of FIG. 3 allows for automated alignment 408 of FIG. 4 of flash and non-flash images to remove motion artifacts.

The system in FIG. 5 includes a decision tree whereby skin conditions are classified based on a set of pre-determined categories. Each node of the decision tree 506-510 of FIG. 5 may be a binary or non-binary classification problem. The classifications in the decision tree comprise whether the wound is “light” or “dark”, the general shape of the condition in terms of aspect ratio and the level of contrast between foreground (skin condition) and background (healthy or intact skin). A number of well established supervised classification algorithms can be used to model these decisions including but not limited to Support Vector Machines (SVM's), soft SVM's, Bayesian classifiers, neural networks, sparse neural networks, nearest neighbor classifiers, multinomial logistic regression and linear regression. Based on current data, it is observed that a soft SVM classifier works best. When a certain threshold of relevant data is accrued by the system, upwards of 5,000 images, an unsupervised classification algorithm can be used to model these decisions including but not limited to spectral clustering, mean shift, auto-encoders or a deep belief network.

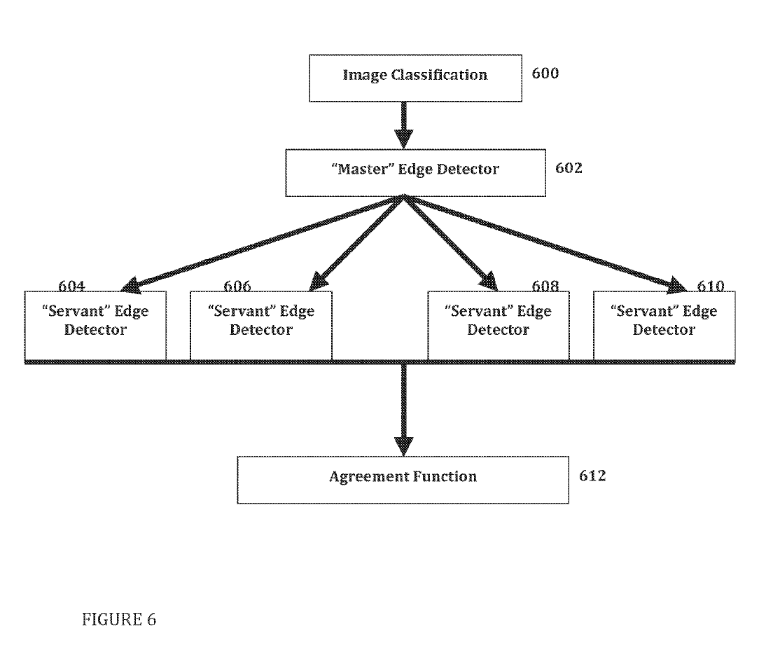

Once the skin conditions have been classified, the expert system of edge detection methods as described by 512-518 in FIG. 5 and as described in further detail by 600-610 in FIG. 6, is applied. In this part of the system, an ensemble of different well established edge detection methods are run on the image in parallel on the image parameters comprising RGB, HSV, YCbCr, texture and range. The ensemble is led by a “master method” 602 and followed by a set of “servant methods” 604-610. The master method 602 is applied more times than each of the servant methods 604-610 and the choice of master method is dictated by the classification of the skin condition as described in the decision tree 506-510 of FIG. 5.

Any methods of edge detection that involve the evolution of a level set are all initialized from different initial spatial coordinates so as to provide variability in results between methods. Said method of initialization allows the different level set methods to evolve according to different image-based gradients thus imposing variation on the level set-based results. This combination of differently initialized level sets reduces the stochastic element associated with choice of initial level set.

The methods of edge detection described in detail applied to the wound, as described in FIG. 6, comprise distance regularized level set evolution (DRLSE) initialized outside the skin condition, DRLSE initialized inside the skin condition, Chan Vese initialized outside the skin condition, Chan Vese initialized inside the skin condition, K Means Algorithm, Soft K Means Algorithm, Gradient Vector Flow (GVF) active contours or simple GVF, Geometric Active Contours, Fuzzy Edge Detection, grabCut, gPb-owt-ucm, Curfil and a convolutional neural network.

Once each of the master methods 602 and servant methods 604-610 are complete an agreement function 612 in FIG. 6 is applied to the combined output of the edge detection methods of FIG. 6. This agreement function 612 takes a weighted vote of each of the pixel masks that the aforementioned edge detection methods created. The weights assigned to each of the edge/boundary detection methods during the vote are assigned based on first and second order characteristics of the skin condition as they relate to an image training set.

Next, the system uses 522 in FIG. 5 an unsupervised clustering technique to segment the wound into different discrete regions. The process involves using a segmentation algorithm comprising K Means Clustering, soft K Means clustering and a Watershed Transformation. The segmentation uses image parameters comprising RGB, HSV, texture, range and histogram of gradients.

The output of the segmentation algorithm are a series of submasks within the initially segmented mask. Each sub-mask is then classified using k bagged neural networks where k is an integer between 50 and 100 as in 524 of FIG. 5. Tissue types classified comprise granulation, slough, necrosis, epithelium, caramelized tissue, bone, tendon, blister, callous, rash, tunneling, undermining and drainage. Using the reference object 300 in FIG. 3, this method is able to calculate the percentage composition of each of the different tissues within the skin condition as well as the area of each of these regions.

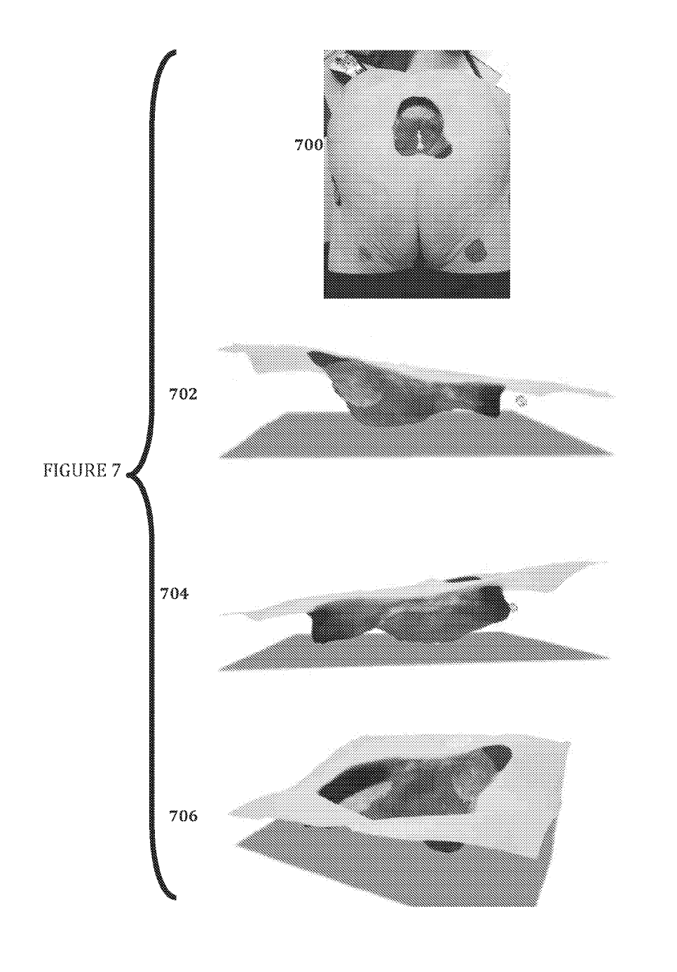

In addition, the system also includes a method for creating a 3D reconstruction of a 2D surface shown by 702-706 in FIG. 7. This method involves taking a short video of the surface of the skin condition with a reference object such as 300 in FIG. 3 being in each frame of the video.

The system uses externally developed software by Trnio, inc. to reconstruct a 3D surface 702-706 of the skin condition by performing mosaicking of the various frames captured in the video using various surface features such as the reference object to facilitate this 3D stitching.

After constructing the 3D surface of the skin condition, the edges of the 3D surface below the base, i.e. the “depth” edges from the ground level slice, clearly illustrated in 702 of FIG. 7, can be detected using the same process as described in FIG. 5. Using the planar dimension of the reference object 300 from FIG. 3, the actual depth of various parts of the 3D surface can be calculated. Using this depth, and the condition's surface area calculated previously, the system can provide values for the total volume, region-specific volume and tissue-specific volume, i.e. depth of tissues, of the skin condition.

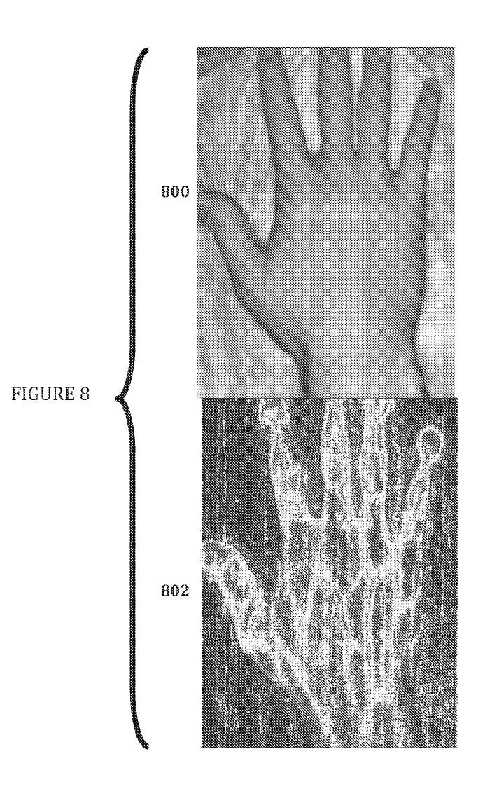

The system also includes a method for identifying a perfusion, or blood flow, profile for the skin condition and the area adjacent to the skin condition as shown by 800-802 of FIG. 8.

This method involves using the aforementioned video of the skin condition and performing a temporal superpixel analysis and spatial decomposition of each of the sequential frames in the video acquired. Once the output of this analysis is amplified, the blood flow to the skin condition and the area surrounding the skin condition can be visualized as in 802 of FIG. 8. The system allows the pace of this visual output to be adjusted manually.

The system also includes a module for calibrating a region with analyzed perfusion to a Laser Doppler Image of the same region. In this process, the color profile of each of the individual frames is analyzed by assessing the regional parameters comprising RGB, HSV, texture and range and comparing these values to the relative perfusion units (RPU) profile of the Laser Doppler Image. Each time a region is manually analyzed, the data is pooled and stored in a database. Each time a new photo is analyzed, the system appropriately queries the database and assigns an RPU value to each region of the image as shown by 802 in FIG. 8.

The front end of the software is a point-of-care data collection engine that allows users to log in using a credentials-based authentication as in 904 of FIG. 9. Options for this data collection engine comprise a mobile phone, tablet and a digital camera combined with a computer with a portable or non-portable workstation.

The point-of-care user, which may be a nurse, aid, physician or patient, can then collect patient consent by reading a script and inputting their digital signature as in 906 in FIG. 9. The aforementioned provider can then collect essential patient information by updating fields based on dropdown menus that contain information pertaining to the specific skin condition. While this data does not directly contribute to the aforementioned image analysis, once it is collected it is mined in a database for future patient tracking.

To give users the ability to accurately report the location of the skin condition, one screen of the data collection engine is equipped with a 3D, rotatable image of a mammalian body as shown in 910 in FIG. 9. Once an area is manually selected, the area becomes highlighted. This selection is given a human readable label and is transmitted to the secure storage area 104 in FIG. 1, where matched with the appropriate patient information and eventually accessed by the a central, ubiquitously accessible web-based portal 112 in FIG. 1.

The user is able to acquire images and a video of the skin condition using the data collection engine as shown by 912-916 and 918-922 in FIG. 9. The user is given the option to draw a box 914 in FIG. 9 around the skin condition after taking the image to guide the image analysis.

The software also provides the option to overlay a semi-transparent image of the skin condition from the previous encounter over the photo-taking device to facilitate image acquisition and tracking of the condition.

For the video capture, a 10 second visible light video is collected. After the video is taken, the data collection engine relays the output of the video capture back to the user. This process is repeated depending on the number of discrete areas affected by the skin conditions on each the user desires to capture and analyze. The user is able to conditionally add discrete areas affected by the aforementioned skin condition at the end of the documentation system on the “send data page” 928 of FIG. 9.

The user also has the opportunity to report patient treatment information, patient skin condition characteristics and any other notes as in 924-926 of FIG. 9. When the user presses “Send Report” on the final page 928 in FIG. 9 the patient image data collected between 912-916 in FIG. 9, video data collected between 918-922 in FIG. 9 and the label associated with the shaded 3D drawing collected in 910 in FIG. 9 to the secure storage area 104 in FIG. 1. Information about the patient is simultaneously sent to the database 104, specifically 106, in FIG. 9. Additionally, information about the patient is automatically compiled into a Portable Document Format (PDF) document and emailed automatically to the emails specified in 904 of FIG. 9. The image and video data sent to the secure storage area is matched with its corresponding patient data by the server component.

Once the image and video data arrives at the secure storage area 104 in FIG. 1 the image analysis node 102 in FIG. 1 automatically performs the aforementioned analysis on the images and videos in the storage area. The output of this analysis comprises size and composition characteristics as well as metadata specifying coordinates for overlay mapping. This data is then returned to the data collection engine so that the user can inspect the annotated output of the image and video analysis. In the case of metadata output, the data collection engine performs automatic image mapping to visually display the output of the image analysis. The user has the ability to reacquire the images and video if not satisfied with the output of the image and video analysis.

Once the user exits out of the data collection engine, any data collected by the user is automatically and immediately deleted from the device hosting the data collection engine.

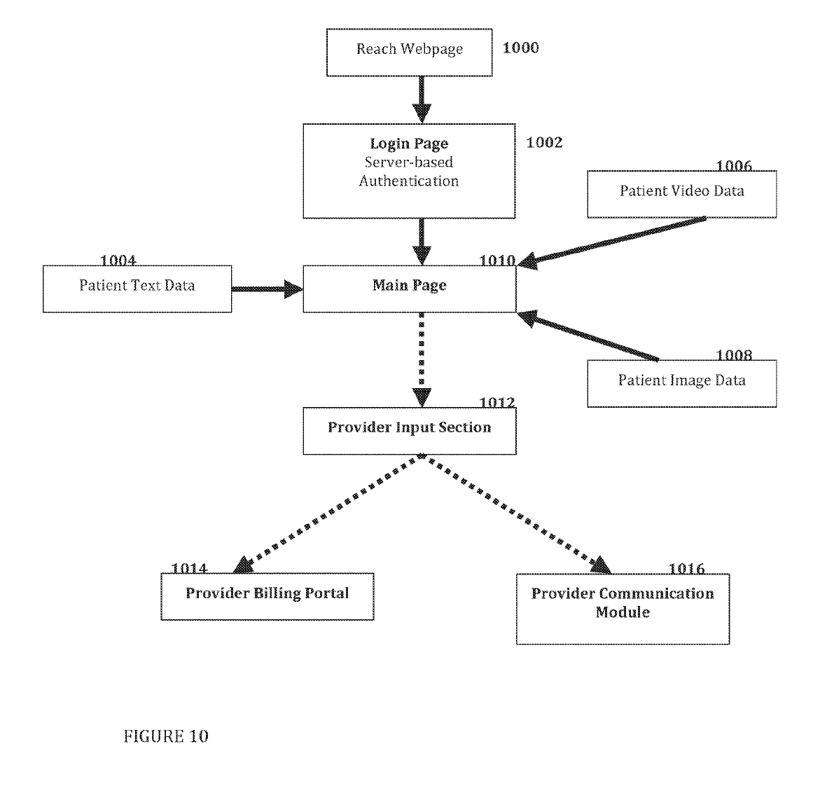

The exemplary embodiment of the system includes an ideal design of a central web portal described in FIG. 10, which can be accessed on any device that has access to the Internet including but not limited to mobile phones, portable and non-portable workstations and tablets.

After all of the data received at the phone, including patient data, images, video and analysis, is matched at the server side, the central web portal 112 in FIG. 1 accesses all of this information and presents it visually for the user. In the case of the central portal, the potential users comprise physicians, nurses, aids or administrators. To access the central portal, the user must be authenticated shown by 1000 in FIG. 10. Authentication credentials are provided and stored securely in the database 104, specifically 106, in FIG. 9.

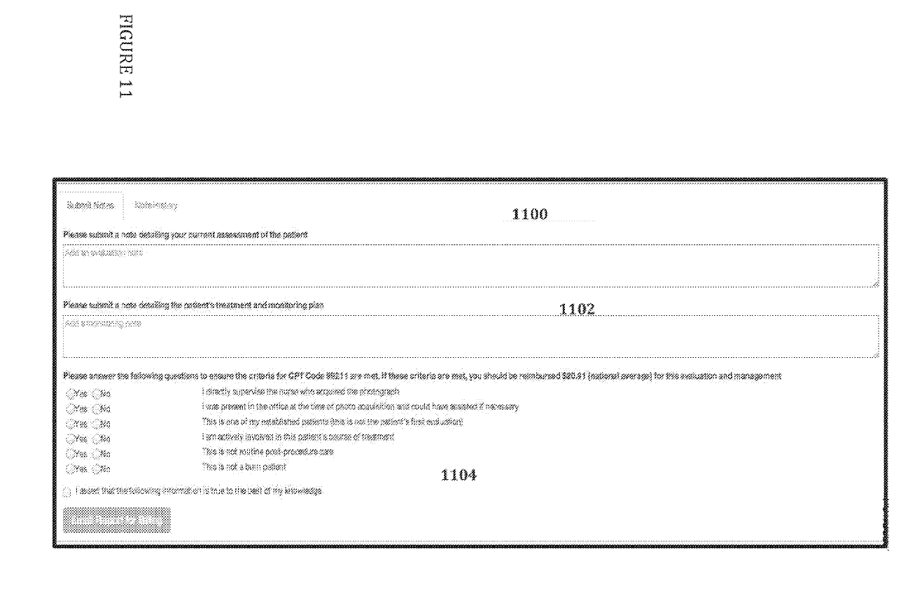

The web portal allows providers to track the progress of all of their patients' skin conditions. This is done by providing both a time lapse image sequence of the digitally depicted progression of the condition as well as a longitudinal graph depicting the progress of the patient's condition on the main page 1010 of FIG. 11.

Using the aforementioned reference object, the software performs automatic scaling of each image in the time lapse in order to standardize and facilitate serial viewing of the skin condition. This is done by collecting and storing the actual length and width of the reference object in units of pixels from the first image collected for a specific patient's skin condition and keeping these values constant for all of the images of said patient's condition.

Once the web portal is accessed, the user can view all of the patients in the user's care at 1010 in FIG. 10. The user also has access to a rich depth of patient information comprising the patient's name, wound etiology, wound bed assessment, pain, odor, pressure ulcer stage, protocols and therapies, start of care, healthcare plan and point-of-care provider name. All of this information is sorted appropriately by the database 104 in FIG. 1.

At this stage, the output of the image analysis and video analysis is displayed to the user of the central portal 112 of FIG. 1 and is matched with the appropriate patient by the database 104 in FIG. 1. The portal also gives the user the ability to adjust the output of the image and video analysis manually if not satisfied with the initial output as in 1012 of FIG. 10. The numerical data fields on the main page 1010 will then be updated automatically corresponding to the user input. The user can also update the patient protocols and therapies directly on the central portal in FIG. 10 to assist coordination of care. The user can also communicate directly with other users on the central portal as in 1016 of FIG. 11.

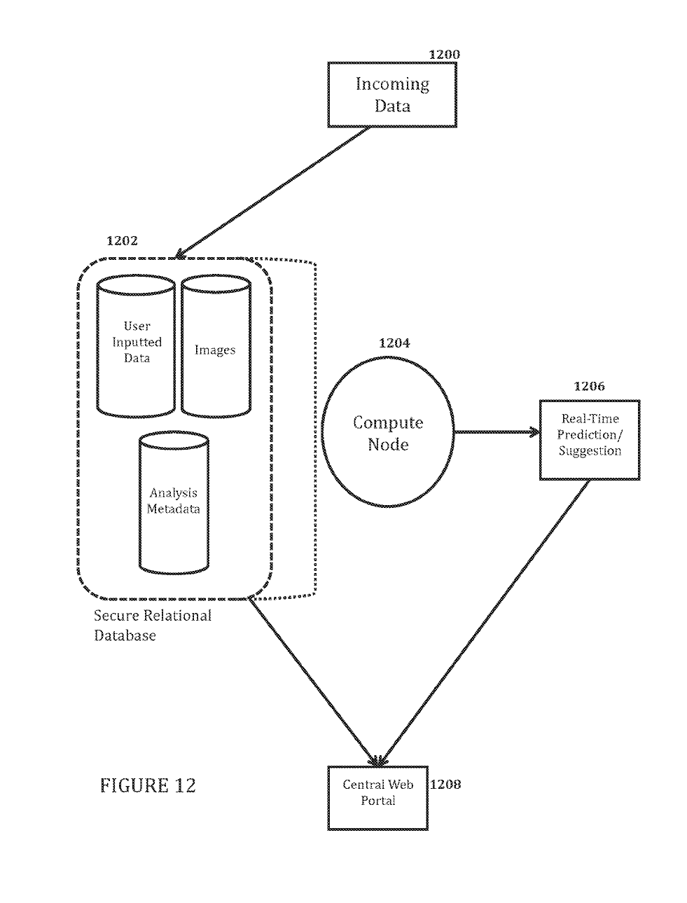

The ideal embodiment of the central portal has an exemplary billing portal shown by FIG. 11 that users of the central portal can use to be reimbursed for using the central portal. The exemplary billing portal also contains a field 1100 in FIG. 12 for the user to enter an evaluation and management note about the patient.

Once the user completes this decision pathway 1104 and fills in the text field(s) 1102 in FIG. 12, the portal automatically generates an American National Standards Institute (ANSI) 837 message including the portal user's insurance information, the patient's healthcare information and the dollar amount requested based on the reimbursement code designated by the central portal. This ANSI 837 message is then automatically relayed to an insurance clearing house.

The ideal embodiment of the central web portal is able to then automatically receive an ANSI 835 message from the clearing house as it relates to the ANSI 837 message that was generated. The central portal can parse the information provided by the ANSI 835 message and relays it to the database 104 in FIG. 1 where it is stored.

The ideal embodiment of the system includes an exemplary predictive analysis engine 1204 in FIG. 12 that performs automated analysis on patient progress based on the serial results of the image and video analysis and compares this analysis to the patient treatment data. The predictive analysis engine 1204 in FIG. 12 is built using established machine learning algorithms comprising support vector machines (SVMs), soft SVMs, neural networks, sparse neural networks, artificial neural networks, decision trees, Cox regression and survival analysis, logistic regression, Bayesian classifiers and linear regressions. The ideal embodiment of the predictive analytics engine uses one or more of the aforementioned algorithms combined with a large, curated data set to predict future patient skin condition progress and suggest treatments based on this prediction.

Once the predictive analysis is complete, the results are stored on the database where they are eventually relayed appropriately to the central web portal 1208 in FIG. 12 so that the user of the central web portal can view the suggestions provided.

It is understood by one of ordinary skill in the art that at least certain variations of the disclosed technology not explicitly described above are still encompassed within the spirit of this disclosure. Hence, the scope of this disclosure extends to at least these variations as understood by one of ordinary skill in the art.

What is claimed is:

1. A method for assessing progress of changes over time to a skin condition that is visible on a mammalian subject, comprising:
obtaining and processing an electromagnetic image of the skin condition in successive iterations at successive times, to characterize the skin condition according to a set of parameter values at each of the successive times, wherein differences in respective said parameter values at the successive times represent said progress of changes;
wherein each iteration includes placing at least one visual reference model on the subject in a region of the skin condition, the reference model having known objective visual characteristics;
collecting at least one image of the region of the skin condition so as to obtain a visual recording representing both the skin condition and the reference model, wherein the at least one image is collected from a perspective angle and distance and at lighting conditions that are at least partly variable from one of the iterations to another;
normalizing the visual recording representing both the wound and the reference model such that an image of the reference model in the visual recording conforms to the known objective visual characteristics of the reference model, thereby also normalizing an image of the wound in the visual recording;
comparing the respective parameter values at the successive times using the image of the wound in the visual recording as thereby normalized.

2. The method of claim 1, wherein the objective visual characteristics include a known shape, a known color characteristic and a known size and said normalizing comprises transforming the visual recording representing both the wound and the reference model to produce a normalized view in which the reference model conforms to said known shape, color characteristic and size.

3. The method of claim 2, wherein the normalized view represents a plan view of the region of the wound, with a shape and color characteristic confirming to the objective visual characterizes and with a known scale relationship to the known size.

4. The method of claim 3, wherein the color characteristic includes at least one of a luminance/saturation/hue characteristic and a luminance/color difference characteristic.

5. The method of claim 1, further comprising segmenting the image of the wound as normalized and comparing said respective parameter values for segments of the image.

6. The method of claim 1, further comprising assessing blood perfusion in tissues associated with the wound, from selected said parameter values taken from at least one of the optical images of the wound.

7. The method of claim 6, further comprising obtaining and processing a video image of the wound and analyzing a plurality of frames in the video image during at least one of the successive iterations for assessing said blood perfusion.

8. The method of claim 7, wherein said analyzing of the plurality of frames includes temporal super pixel analysis and spatial decomposition.

9. The method of claim 7, further comprising reassessing said blood perfusion during said successive iterations at successive times.

10. The method of claim 1, further comprising generating a three dimensional reconstruction of the wound from plural images of the wound obtaining during at least one of the iterations.

11. The method of claim 10, wherein the three dimensional reconstruction includes determining surface topography of the wound and inferring a depth of tissues.

12. A method for assessing progress of changes over time to a skin condition that is visible on a mammalian subject, comprising:
obtaining and processing an electromagnetic image of the skin condition in successive iterations at successive times, to characterize the skin condition according to a set of parameter values, wherein differences in respective said parameter values over time represent said progress of changes;
collecting at least one image of the region of the skin condition so as to obtain a visual recording representing the skin condition at each of the successive iterations, wherein the images at the successive iterations
normalizing the images of the successive iterations for perspective angle, distance, luminance and color difference, at least in the region of the skin condition;
comparing the respective parameter values at the successive times using the image of the wound in the visual recording as thereby normalized, to produce at least one level set having a series of said parameter values proceeding along a path intersecting at least part of the region of the skin condition.

13. The method of claim 12, wherein the successive iterations are at irregular intervals.

14. The method of claim 12, comprising plural said level sets initialized from different spatial coordinates.

15. The method of claim 14, comprising comparing values along the plural level sets and distinguishing areas within and outside of a wound based on a threshold number of the level sets meeting a predetermined criterion.
